(19) United States
(12) Patent Application Publication
(10) Pub. No.: US 2012/0219637 A1
(43) Pub. Date: Aug. 30, 2012

(54) PHARMACEUTICAL COMPOSITIONS OF LANTHANUM CARBONATE AND PROCESS FOR THE PREPARATION THEREOF

(76) Inventors: Dhore Aniket, Maharashtra (IN); Ranjan Pradhan Manas, Orissa (IN); V. Sathyanaryana, Mumbai (IN); Rampal Ashok, Haryana (IN)

(21) Appl. No.: 13/504,082

(22) PCT Filed: Oct. 25, 2010
(86) PCT No.: PCT/IN10/00691

§ 371 Date: Apr. 25, 2012

(30) Foreign Application Priority Data
Oct. 26, 2009 (IN) 2470/MUM/2009

(51) Int. Cl. A61K 33/24; A61P 7/00

(52) U.S. Cl. 424/617

(57) ABSTRACT

The present invention relates to compact pharmaceutical compositions of lanthanum carbonate comprising diluents in an amount ranging from about 30% w/w to about 40% w/w of the composition.

### FIELD OF THE INVENTION

The present invention relates to pharmaceutical compositions comprising lanthanum carbonate & process for the preparation of the same.

### BACKGROUND OF THE INVENTION

Lanthanum is a rare earth element of transition group IIIB of the periodic table. It was discovered by Carl Gustaf Mosander in 1839. Its name derived from the Greek lanthanein, meaning “to be concealed”, indicating that it is difficult to isolate. Lanthanum carbonate, La₂(CO₃)₃, salt formed by lanthanum(III) cations and carbonate anions, is a non-calcium, non-aluminum containing phosphate binder. It is an ore of lanthanum metal, along with monazite with an atomic number 57 and an atomic weight of 139.

Lanthanum carbonate binds phosphate optimally at pH 3-5, while retaining binding activity across the full range of pH 1-7. It is, therefore, able to bind phosphate efficiently at the low pH of the stomach as well as the higher values in the duodenum and jejunum, unlike calcium carbonate. Thus, following administration with food, lanthanum carbonate forms a compound of low aqueous solubility (i.e., lanthanum phosphate) which does not readily pass through the lining of the gastrointestinal tract into the blood. As a consequence, phosphate absorption from the diet is decreased. Dietary phosphate bound to lanthanum carbonate is excreted in the feces, with a resultant lowering of serum phosphorus and urinary phosphorus excretion. In United States., the FDA approved Fosrenol® (lanthanum carbonate) indicating reduction in serum phosphate levels in patients with end-stage renal disease (ESRD), is now available to patients having appeared on the market during early 2005. Apart from medicinal, lanthanum carbonate is also used for the tinting of glass, for water treatment, or as a catalyst for hydrocarbon cracking.

U.S. Pat. No. 5,968,976 discloses that selected lanthanum carbonate hydrates with 3 to 6 molecules of water of crystallization, may be administered into the gastrointestinal tract, to treat hyperphosphataemia in patients with renal failure.

U.S. Pat. No. 7,381,428 & U.S. Patent application No. 20080187602A1 disclose stabilized lanthanum carbonate compositions containing a monosaccharide or disaccharide stabilizing agent and subjects having hyperphospliatemia can be treated by administering a pharmaceutical composition containing a therapeutically effective amount of the stabilized lanthanum carbonate formulation. The inventions also disclose that the amount of diluents is from about 40 to about 80% w/w of the formulation. The dosage forms disclosed in this invention are bulky, having 4168 mg, 3126 mg, 2084 mg and 1042 mg of total weights for 1000 mg, 750 mg, 500 mg and 250 mg equivalent lanthanum base strength dosage forms respectively, due to the higher amounts of diluents used.

U.S. Pat. No. 7465465 & U.S. Patent application No. 20090017133A1 relate to a chewable lanthanum formulation comprising a pharmaceutically effective amount of a lanthanum compound; and at least one chewable pharmaceutically acceptable excipient. This invention also discloses a pharmaceutical formulation of a lanthanum compound in a tablet or in a powder form produced by a process comprising the steps of: a) powder blending the lanthanum compound and at least one pharmaceutically acceptable excipient in a mixer to form a mixture; or b) powder blending the lanthanum compound and excipients, compressing the resulting combination into a slug material or roller compacting the resulting combination into a strand material, and milling the prepared material into a free flowing mixture; and c) compressing the resulting mixture into a tablet or filing up the resulting mixture in a appropriate container. This patent & application also disclose the use of monosaccharide or disaccharide chewable pharmaceutically acceptable excipients in an amount from 20 to 80% w/w of the formulation. However, in spite of the seemingly low amounts of the diluents that may be used in this patent & application, the dosage forms disclosed in this invention are bulky, having 4168 mg, 3126 mg, 2084 mg and 1042 mg of total weights for 1000mg, 750 mg, 500 mg and 250 mg equivalent lanthanum base strength dosage forms respectively.

U.S. Patent No. 20080125394A1 relates to medicaments useful for reducing phosphorus serum level, especially in those subjects affected from hyperphosphatemia. It discloses pharmaceutical compositions comprising a phosphorus compound binding agent and at least one pharmaceutically acceptable vehicle and/or excipients, to be administered by oral route in fasting periods, in order to absorb phosphorus compounds from fluids of the enteric tract, especially from saliva.

In the above cited prior art patents/patent applications, high unit doses of lanthanum carbonate provide a bulky dosage form. Also the commercially available tablets in United States i.e. Fosrenol® are typically bulky, for example, about 4160 mg, about 3120 mg about 2080 mg for 1000 mg, 750 mg and 500 mg equivalent lanthanum base strength dosage forms respectively. Bulky dosage forms pose difficulty in swallowing and especially when presented as a chewable tablet, it becomes a potential barrier for their use considering patient compliance and handling. Moreover, it necessitates effective process optimization with facilitated die filling and blend lubrication to ensure efficient tablet production at plant scale

The present invention has been made in view of overcoming the aforementioned problems of the prior art. We have surprisingly found that using lesser amount of diluents provides small sized or compact tablets having both patient compliance and adaptability for formulation processing. It was also found, that in spite of the use of lesser amount of diluents, the compact pharmaceutical compositions of lanthanum carbonate were stable as well as therapeutically equivalent to commercially available, more bulky, Fosrenol® tablets.

OBJECT OF THE INVENTION

It is an object of the present invention to provide pharmaceutical compositions of lanthanum carbonate comprising diluents in an amount ranging from about 30% w/w to about 40% w/w of the composition.

It is an object of the present invention to provide compact pharmaceutical compositions of lanthanum carbonate comprising diluents in an amount ranging from about 30% w/w to about 40% w/w of the composition.

It is an object of the present invention to provide stable compact pharmaceutical compositions of lanthanum carbonate comprising diluents in an amount ranging from about 30% w/w to about 40% w/w of the composition.

It is another object of the present invention to provide compact pharmaceutical compositions of lanthanum carbonate comprising diluents in an amount ranging from about 30% w/w to about 40% w/w of the composition, which are therapeutically equivalent to commercially available lanthanum carbonate tablet in the United States of America i.e. Fosreno®.

It is yet another object of the present invention to provide a process to prepare compact pharmaceutical compositions of lanthanum carbonate comprising diluents in an amount ranging about 30% w/w to about 40% w/w of the composition.

At least one of the preceding objects is met, in whole or in part, by a process providing the use of diluents in an amount of from about 30% w/w to about 40% w/w of the composition to prepare compact pharmaceutical compositions of lanthanum carbonate.

### SUMMARY OF THE INVENTION

The present invention relates to pharmaceutical compositions of lanthanum carbonate comprising diluents in an amount ranging from about 30% w/w to about 40% w/w of the composition.

The present invention relates to compact pharmaceutical compositions of lanthanum carbonate comprising diluents in an amount ranging from about 30% w/w to about 40% w/w of the composition.

The present invention relates to compact pharmaceutical compositions of lanthanum carbonate comprising diluents in an amount ranging from about 30% w/w to about 40% w/w of the composition, which are stable.

The present invention also relates to compact pharmaceutical compositions of lanthanum carbonate comprising diluents in an amount ranging from about 30% w/w to about 40% w/w of the composition, which are therapeutically equivalent to commercially available lanthanum carbonate tablet in the United States of America i.e. Fosrenol®.

Accordingly, the present invention relates to a process of preparing compact pharmaceutical compositions of lanthanum carbonate comprising adding diluents in an amount ranging from about 30% w/w to about 40% w/w of the composition and optionally adding solubilizers.

### DESCRIPTION OF THE INVENTION

Before the present compositions and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context dearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the step” includes reference to one or more step and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “pharmaceutical compositions” as used herein includes solid oral dosage forms such as powder, beads, granules, tablets, capsules, chewable compositions and the like, that in turn may be prepared by conventional methods known to a person skilled in the art.

The term “compact” as used herein refers to a pharmaceutical compositions of lanthanum carbonate which are weighing less than about 4000 mg, about 3000 mg, about 2000 mg and about 1000 mg with diameter of less than about 21 mm, about 19 min, about 17 mm and about 14 mm for 1000 mg, 750 mg, 500 mg and 250 mg equivalent lanthanum base strength compositions respectively.

The term “stable” as used herein refers to chemical stability of lanthanum carbonate in solid dosage forms wherein there is no change in assay values and dissolution profile when kept at 40° C./75% RH for 3 months.

The term “therapeutically equivalent” as used herein refers to the pharmaceutical composition of the present invention having a similar degree of phosphate binding as that of commercially available Fosrenol® tablets as determined by invitro phosphate binding studies as well as having a similar dissolution profile.

We have surprisingly found that using lesser amount of diluents provides small sized or compact tablets having both patient compliance and adaptability for formulation processing. It was also found, that in spite of the use of lesser amount of diluents, the compact pharmaceutical compositions of lanthanum carbonate were stable as well as therapeutically equivalent to commercially available, more bulky, Fosrenol® tablets.

The present invention relates to pharmaceutical compositions of lanthanum carbonate comprising diluents in an amount ranging from about 30% w/w to about 40% w/w of the composition.

The present invention relates to compact pharmaceutical compositions of lanthanum carbonate comprising diluents in an amount ranging from about 30% w/w to about 40% w/w of the composition.

The present invention relates to compact pharmaceutical compositions of lanthanum carbonate comprising diluents in an amount ranging from about 30% w/w to about 40% w/w of the composition, which are stable.

The present invention also relates to compact pharmaceutical compositions of lanthanum carbonate comprising diluents in an amount ranging from about 30% w/w to about 40% w/w of the composition, which are therapeutically equivalent to commercially available lanthanum carbonate tablet in the United States of America i.e. Fosrenol®).

Accordingly, the present invention relates to a process of preparing compact pharmaceutical compositions of lanthanum carbonate comprising adding diluents in an amount ranging from about 30% w/w to about 40% w/w of the composition and and optionally adding solubilizers.

As used herein, lanthanum carbonate refers to all polymorphs and hydrated forms of lanthanum carbonate and anhydrous lanthanum carbonate of the general formula:

La₂(CO₃)₃.xH₂O

where x has a value from 0 to 10, preferably x has an average value of 8. For example, the stable pharmaceutical composition of the invention may include from about 30% w/w to about 70% w/w of lanthanum carbonate by weight of the composition.

Lanthanum carbonate has a tendency to degrade via decarboxylation to lanthanum hydroxycarbonate as shown:

La2(CO3)3+nH2O→2LaOHCO3+CO2+(n−1)H2O

This process is accelerated in the presence of moisture or heat and appears to be self-catalyzing. Hence, even a very small amount of lanthanum hydroxycarbonate in lanthanum carbonate compositions causes rapid and excessive degradation. Further, conditions sufficient to bring about decarboxylation of these materials may be present during their manufacture as well as during storage in a formulated or unformulated state.

Diluents are added in the composition of the present invention to increase the bulk volume of the powder to facilitate granulation or compression and may also function as sweetening agents. The present invention uses diluents selected from the group comprising powdered cellulose, microcrystalline cellulose, silicified microcrystalline cellulose, lactose, starch, dibasic calcium phosphate, tribasic calcium phosphate, calcium carbonate, dextrates, dextrin, dextrose, kaolin, magnesium carbonate, magnesium oxide, sugars such as sucrose; sugar alcohols such as maltitol, mannitol, sorbitol, erythritol; and the like and mixtures thereof. A preferred diluent is maltitol, which may also act as a stabilizing agent. One or more diluents can be present in the composition. The diluents may be present in the compositions in an amount ranging from about 30% w/w to about 40% w/w, and preferably from about 35% w/w to about 40% w/w.

The pharmaceutical compositions of the present invention may further comprise solubilizers known in the art. The solubilizers may be selected from the group comprising poloxamer, sodium lauryl sulfate, Tweens, Spans, lecithin, Polysorbate, polyethylene glycol, cyclodextrin, Gelucier, docusate sodium, polyvinylpyrrolidone, hydroxypropyl methylcellulose, Transcutol (diethylene glycol monoethyl ether) and the like and mixtures thereof Preferred solubilizers are poloxamers, particularly poloxamer 407. The solubilizers employed in the present invention additionally affect drug release and thus provide equilibrium for the use of lesser amount of diluents for the purpose of the present invention. The solubilizers may be used in the composition in an amount ranging from about 0.1% w/w to about 15% w/w of the composition.

The pharmaceutical compositions of the present invention may further comprise conventional pharmaceutically acceptable excipients. Conventional pharmaceutical excipients include those which function in a dosage form, for example, as a binder, lubricants, glidant, disintegrants, colors and flavors.

Binding agents which may be employed include, but are not limited to, hydroxyethyl cellulose, hydroxypropyl cellulose, hydroxypropyl methylcellulose, carbomers, carboxymethylcellulose sodium, Hydroxypropyl β-cyclodextrin, dextrin, methylcellulose, shellac, zein, gelatin, polymethacrylates, polyvinyl pyrrolidone, pregelatinized starch, potato starch, sodium alginate, gums, synthetic resins and the like and the mixtures thereof. The binding agent may be present in the composition in an amount ranging from about 2% w/w to about 15% w/w, preferably from about 5% w/w to about 10.5% w/w.

Disintegrants that may be used in the composition include, but are not limited to, cross povidone, cross carmellose sodium, sodium starch glycolate and the like and the mixtures thereof. The disintegrants may be present in the composition in an amount ranging from about 0.001% w/w to about 10% w/w, preferably from about 0.05% w/w to about 5% w/w.

Lubricants that may be used in the composition include, but are not limited to, sodium stearyl fumarate, zinc stearate, calcium stearate, stearic acid or mixtures thereof; providing the stable pharmaceutical compositions comprising lanthanum carbonate. Lubricants of the present invention, particularly, sodium stearyl fumarate is relatively inert and therefore avoids incompatibilities with lanthanum carbonate which may results in a better stability. The lubricants of the present invention may be used in the stable pharmaceutical composition of the present invention in amounts ranging from about 0.1% w/w to about 5% w/w, preferably from about 1% w/w to about 3.5% w/w of the composition.

Optionally, antioxidants may also be incorporated in the composition to enhance its storage stability, for example, ascorbic acid, alpha tocopherol or butylated hydroxyanisole. One or more antioxidants can be present in the composition. The total antioxidant amount can be preferably from about 0.001% to about 0.1%, and more preferably from about 0.005% to 0.05% by weight of the composition.

As discussed above, the pharmaceutical compositions of the present invention may be prepared by conventional techniques such as dry admixing, wet granulation, dry granulation or direct compression. In dry admixing, lanthanum carbonate is mixed with diluents, optional solubilizers and various other excipients in a mixer to form a mixture. In wet granulation, lanthanum carbonate is mixed with diluents, optional solubilizers and various other excipients and granulated, followed by screening and drying of the damp mass. The dried mass may be screened, lubricated. Dry granulation can be done by two processes: (1) slugging, which involves mixing the lanthanum carbonate with diluents, optional solubilizers and the other excipients, slugging, dry screening, or (2) roller compaction process. The granules obtained by the said granulating processes, can again be mixed with optional solubilizers & either be filled in capsules or compressed into tablets. Direct compression involves compressing tablets directly from the physical mixture of lanthanum carbonate, diluents, optional solubilizers and the other excipients. Alternatively the pharmaceutical compositions of the present invention may be obtained by preparing placebo granules comprising the diluents, optional solubilizers and other pharmaceutically acceptable excipients, and mixing these with lanthanum carbonate to obtain a blend, which may be compressed into tablets. These methods provide compositions of lanthanum carbonate that are stable.

The following examples are intended to illustrate the scope of the present invention in all its aspects but not to limit it thereto.

### EXAMPLE 1

The compact pharmaceutical composition of the present invention may be prepared as given in Table 1.
Label Claim: Each chewable tablet contains Lanthanum carbonate Hydrate Equivalent to Elemental Lanthanum of respective strength.

TABLE 1

| | Strengths | | | | |
|---|---|---|---|---|---|
| Name of | 1000 mg | 750 mg | 500 mg | 250 mg | |
| Ingredients | (mg/tab) | (mg/tab) | (mg/tab) | (mg/tab) | % w/w |
| Lanthanum | 2162.02 | 1621.515 | 1081.01 | 540.505 | 60.06% |
| carbonate | | | | | |
| octahydrate | | | | | |
| Maltisorb P | 1290.98 | 968.235 | 645.49 | 322.745 | 35.86% |
| 200(Maltitol) | | | | | |
| crospovidone | 20.00 | 15 | 10 | 5 | 0.56% |
| Poloxamer 407 | 100.00 | 75 | 50 | 25 | 2.78% |
| Sodium Stearyl | 27.00 | 20.25 | 13.5 | 6.75 | 0.75% |
| Fumarate | | | | | |
| Total tablet wt | 3600 mg | 2700 mg | 1800 mg | 900 mg | — |

Lanthanum Carbonate Octahydrate (30 #), Maltitol (30 #) and crospovidone (40 #) were mixed for 25 minutes in a blender. Poloxamer was sifted through 40# and mixed with it for further 5 min. The above dry mix was compacted by using compactor. After compaction, the flakes were milled by using Multimill of 2.5 nun screen. For extra granular addition, poloxamer 407 and Cross Povidone were sifted through 40 # and mixed with sized granules. Sodium Stearyl Fumarate was sifted through 40# and mixed with it for 5 min. The above obtained blend was compressed with their respective punches.

The compact pharmaceutical compositions as prepared in Table 1 were subjected to tablet evaluation tests like weight variation and tablet dimension like diameter and thickness and this was compared with the marketed composition in US i.e. Fosrenol®. The results are given in Table 2.

TABLE 2
Lanthanum Tablets

| wt of | | | | | | | | |
|---|---|---|---|---|---|---|---|---|
| individual | | | | | | | | |
| tablets | Fosrenol ® | Test | Fosrenol ® | Test | Fosrenol ® | Test | Fosrenol ® | Test |
| (in mg) | 1000 mg | 1000 mg | 750 mg | 750 mg | 500 mg | 500 mg | 250 mg | 250 mg |
| 1 | 4237.8 | 3654 | 3113 | 2725.1 | 2115 | 1812.6 | not | 908.2 |
| 2 | 4184.3 | 3625 | 3115 | 2714.9 | 2095.1 | 1819.2 | available | 905.6 |
| 3 | 4162.8 | 3651 | 3121 | 2719.6 | 2124.5 | 1805.1 | | 910.1 |
| Average | 4194.97 | 3643.33 | 3116.33 | 2719.87 | 2111.53 | 1812.30 | | 907.70 |
| wt | | | | | | | | |
| Diameter | | | | | | | | |
| (in mm) | | | | | | | | |
| 1 | 22.17 | 20.07 | 20.16 | 18.04 | 18.17 | 16.03 | | 13.01 |
| 2 | 22.14 | 20.05 | 20.17 | 18.05 | 18.18 | 16.05 | | 13.04 |
| 3 | 22.16 | 20.07 | 20.16 | 18.03 | 18.16 | 16.05 | | 13.05 |
| Thickness | | | | | | | | |
| (in mm) | | | | | | | | |
| 1 | 6.99 | 6.49 | 6.3 | 6.1 | 5.09 | 5.18 | | 3.9 |
| 2 | 6.93 | 6.52 | 6.28 | 6.12 | 5.07 | 5.15 | | 3.93 |
| 3 | 6.95 | 6.54 | 6.28 | 6.14 | 5.09 | 5.11 | | 3.92 |

It can be seen from the above results, that the tablets of the invention weigh less, have a smaller diameter and lesser thickness than the marketed tablets. It can also be seen that the tablets of the invention are compact.

The compact pharmaceutical composition as prepared in table 1 were subjected to dissolution. The results are given in Table 3.
Media 0.25N HCl

TABLE 3

| | | Fosrenol ® Tablets | Test |
|---|---|---|---|
| Sr. No. | Time in min | (% drug release) | (% drug release) |
| 1. | 30 | 93.16 | 99.96 |
| 2. | 45 | 104.36 | 99.8 |

### EXAMPLE 2

The compact pharmaceutical composition of the present invention may be prepared as given in Table 4.

TABLE 4

| Name of Ingredients | Quantity | |
|---|---|---|
| Intra Granular | (Mg/Tab) | % w/w |
| Lanthanum | 2162.02 | 58.43 |
| carbonate | | |
| octahydrate | | |
| Maltitol | 1340.98 | 36.24 |
| crospovidone | 20 | 0.54 |
| Sodium starch | 100 | 2.70 |
| glycolate | | |
| poloxamer | 50 | 1.35 |
| Sodium Steryl | 27 | 0.73 |
| Fumarate | | |
| Total | 3700 | 100 |

| | |
|---|---|
| Diameter of tablet | 20 mm |

Lanthanum Carbonate Octahydrate, Maltitol, crospovidone, Sodium starch glycolate and poloxamer were sifted through suitable sieve and mixed for appropriate time in a blender. Sodium steryl fumarate was sifted through suitable sieve and mixed with it for 5 min. The above dry mix was slugged by using 22.0 mm flat punch. After slugging, it was deslugged by Multimill using suitable screen. The deslugged granules were mixed for 5 min. The above obtained blend was compressed.

### EXAMPLE 3

The compact pharmaceutical composition of the present invention may be prepared as given in Table 5.

TABLE 5

| Name of Ingredients | Quantity | |
|---|---|---|
| Intra Granular | (Mg/Tab) | % w/w |
| Lanthanum | 2162.02 | 60.06 |
| carbonate | | |
| octahydrate | | |
| Maltitol | 1250 | 34.72 |
| Sodium starch | 127.98 | 3.56 |
| Glycolate | | |
| Sodium Steryl | 50 | 1.39 |
| Fumarate | | |
| Total | 3600 | 100 |

| | |
|---|---|
| Diameter of tablet | 20 mm |

The pharmaceutical composition was prepared by a process similar to that used in examples 1, 2 and 3.

The pharmaceutical composition as prepared in example 3 (table 5) was subjected to dissolution. The results are given below.
Media 0.25 N HCl

| | | Composition of |
|---|---|---|
| | Fosrenol ® Tablet | example -2 |
| Time in min | (% drug release) | (% drug release) |
| 45 min | 103.8 | 96.7 |

### EXAMPLE 4

The compact pharmaceutical composition of the present invention may be prepared as given in Table 6.

TABLE 6

| Name of Ingredients | Quantity | |
|---|---|---|
| Intra Granular | (Mg/Tab) | % w/w |
| Lanthanum | 2162.02 | 58.43 |
| carbonate | | |
| octahydrate | | |
| Maltitol | 1340.98 | 36.24 |
| crospovidone | 50 | 1.35 |
| poloxamer | 120 | 3.24 |
| Sodium Steryl | 27 | 0.73 |
| Fumarate | | |
| Total | 3700 | 100 |

| | |
|---|---|
| Diameter of tablet | 20 mm |

The pharmaceutical composition was prepared by a process similar to that used in examples 1, 2 and 3.

The compact pharmaceutical composition as prepared in example 4 (table 6) was subjected to dissolution. The results are given below.
Media 0.25 N HCl

| | | Composition of |
|---|---|---|
| | Fosrenol ® Tablet | example -3 |
| Time in min | (% drug release) | (% drug release) |
| 45 min | 103.8 | 98.9 |

### EXAMPLE 5

The compact pharmaceutical composition of the present invention may be prepared as given in table 7.

TABLE 7

| Name of Ingredients | Quantity | |
|---|---|---|
| Intra Granular | (Mg/Tab) | % w/w |
| Lanthanum | 2162.02 | 61.77 |
| carbonate | | |
| octahydrate | | |
| Maltitol | 1112.98 | 31.80 |
| crospovidone | 50 | 1.43 |
| poloxamer | 150 | 4.30 |
| Sodium Steryl | 25 | 0.71 |
| Fumarate | | |
| Total | 3500 | 100 |

| | |
|---|---|
| Diameter of tablet | 20 mm |

All items except lubricant were sifted through suitable sieve and mixed in a blender. The above dry mix was compacted by using roll compactor. After compaction, the compacted flakes were broken & sized through appropriate sieve & screen. Lubricant was sifted through and mixed with it for 5 min. The above obtained blend was compressed to give the compact formulation of the invention

The compact pharmaceutical composition as prepared in example 5 (table 7) was subjected to dissolution. The results are given below.
Media: 0.25 N HCl

| | | Composition of |
|---|---|---|
| | Fosrenol ® Tablet | example |
| Time in min | (% drug release) | (% drug release) |
| 45 min | 103.8 | 99.8 |

The compact pharmaceutical composition as prepared in example 5 (table 7) was subjected to accelerated stability study (40°/75% RH) where the assay and dissolution was recorded. The results are given in table 8.

TABLE 8

Composition of example

| | | (% drug release |
|---|---|---|
| Periods | Assay | after 45 minutes) |
| Initial | 100.87 | 99.80 |
| 1M—40°/75% RH | 103.81 | 103.2 |
| 2M—40°/75% RH | 100.0 | 101.20 |
| 3M—40°/75% RH | 102.44 | 100.20 |

The results of the table 8 show that the compact pharmaceutical composition of the present invention is stable.

The compact pharmaceutical composition as prepared in example 5 (table 7) and the reference product i.e. Forsenol were subjected to in-vitro phosphate Binding study. The results are as shown below:

| | |
|---|---|
| T/R | 90% Confidence Interval |

| Ratio | Lower | Upper |
|---|---|---|
| 93.40 | 81.48 | 97.32 |

From in-vitro physiochemical parameters and phosphate binding study, it was demonstrated that the test product i.e the compact pharmaceutical composition of the invention exhibits similar phosphate binding properties to the reference product & would be therapeutically equivalent to the reference product.

Although the invention has been described in terms of particular embodiments and applications, one of ordinary skill in the art, in light of this teaching, can generate additional embodiments and modifications without departing from the spirit of or exceeding the scope of the claimed invention. It should be emphasized that the above-described embodiments of the present invention, particularly any “preferred” embodiments, are merely possible examples of the invention of implementations, merely set forth for a clear understanding of the principles of the invention. Accordingly, it is to be understood that the drawings and descriptions herein are proffered by way of example to facilitate comprehension of the invention and should not be construed to limit the scope thereof.

1. Compact pharmaceutical compositions of lanthanum carbonate comprising diluents in an amount ranging from about 30% w/w to about 40% w/w of the composition.

2. Compact Pharmaceutical compositions of lanthanum carbonate as in claim 1, which are stable.

3. Compact pharmaceutical compositions of lanthanum carbonate as in claim 1, which are therapeutically equivalent to commercially available lanthanum carbonate tablet in the United States of America i.e. Fosrenol®.

4. Compact pharmaceutical compositions as in claim 1, further comprising solubilizers.

5. Compact pharmaceutical compositions as in claim 1, wherein the diluents are selected from the group comprising powdered cellulose, microcrystalline cellulose, silicified microcrystalline cellulose, lactose, starch, dibasic calcium phosphate, tribasic calcium phosphate, calcium carbonate, dextrates, dextrin, dextrose, kaolin, magnesium carbonate, magnesium oxide, sugars such as sucrose; sugar alcohols such as maltitol, mannitol, sorbitol, erythritol; and the like and mixtures thereof.

6. Compact pharmaceutical compositions as in claim 4, wherein the solubilizers are selected from the group comprising poloxamer, sodium lauryl sulfate, Tweens, Spans, lecithin, Polysorbate, polyethylene glycol, cyclodextrin, Gelucier, docusate sodium, polyvinylpyrrolidone, hydroxypropyl methylcellulose, Transcutol (diethylene glycol monoethyl ether) and the like and mixtures thereof.

7. A process of preparing compact pharmaceutical compositions of lanthanum carbonate comprising adding diluents in an amount of from about 30% w/w to about 40% w/w of the composition and optionally adding solubilizers.
